SEQUENCE LISTING (8 sequences)

SEQ ID NO: 1 - DNA, 21 bp, Artificial
  Influenza B Matrix Gene Segment 7 forward primer/probe
tcatcacagarcccytatcag

SEQ ID NO: 2 - DNA, 22 bp, Artificial
  Influenza B Matrix Gene Segment 7 reverse primer/probe
gatctcgctgctctgctatgag

SEQ ID NO: 3 - DNA, 271 bp, Artificial
  Influenza B Matrix Gene Segment 7 Amplicon
tcatcacagagcccttatcaggaatgggaacaacagcaacaaaaaagaaaggcctgattctggctgagagaaaaatgagaagatgtgtgagctttcatgaagcatttgaaatagcagaaggccatgaaagctcagcgctactatactgtctcatggtcatgtacctgaatcctggaaattattcaatgcaagtaaaactaggaacgctctgtgctttgtgcgagaaacaagcatcacattcacacagggctcatagcagagcagcgagatc

SEQ ID NO: 4 - DNA, 24 bp, Artificial
  Influenza B Matrix Gene Segment 7 Amplicon Probe
ttgttgctgttgttcccattcctg

SEQ ID NO: 5 - DNA, 21 bp, Artificial
  Influenze B matrix gene segment 7 forward primer/probe
tcatcacagaacccctatcag

SEQ ID NO: 6 - DNA, 21 bp, Artificial
  Influenza B matrix gene segment 7 forward primer/probe
tcatcacagaacccttatcag

SEQ ID NO: 7 - DNA, 21 bp, Artificial
  Influenza B matrix gene segment 7 forward primer/probe
tcatcacagagcccctatcag

SEQ ID NO: 8 - DNA, 21 bp, Artificial
  Influenza B matrix gene segment 7 forward primer/probe
tcatcacagagcccttatcag

(19) United States
(12) Patent Application Publication
(10) Pub. No.: US 2011/0039715 A1
(43) Pub. Date: Feb. 17, 2011

(54) INFLUENZA B VIRUS DETECTION METHOD AND KIT THEREFOR

(75) Inventors: Masafumi Inoue, Singapore (SG); Timothy Barkham, Singapore (SG)

Correspondence Address: Convergent Law Group LLP, P.O. BOX 1329, MOUNTAIN VIEW, CA, 94042, US

(73) Assignees: AGENCY FOR SCIENCE, TECHNOLOGY AND RESEARCH, Singapore (SG); TAN TOCK SENG HOSPITAL, Singapore (SG)

(21) Appl. No.: 12/516,569

(22) PCT Filed: Nov. 23, 2007
(86) PCT No.: PCT/SG07/00403

§ 371 Date: Nov. 16, 2009

(30) Foreign Application Priority Data
Nov. 27, 2006 (SG) 200608133-5

(51) Int. Cl. C40B 30/04; C07H 21/04; C12Q 1/70

(52) U.S. Cl. 506/9; 536/23.1; 435/5

(57) ABSTRACT

The invention provides oligonucleotide(s) for simple, specific and/or sensitive test(s) for the presence of Influenza virus. In particular, the present invention provides oligonucleotide(s) for test(s) for the Influenza B virus. Kit(s) comprising the oligonucleotide(s) for use as probe(s) and/or primer(s) useful in the test(s) are also provided.

### FIELD OF THE INVENTION

The present invention relates to oligonucleotide(s), method(s) and kit(s) for influenza virus infection detection. In particular, the invention provides a nucleic acid assay and kit for the detection of Influenza B virus.

### BACKGROUND OF THE ART

Influenza B is an infectious disease in man caused by type B strains of the influenza virus, an RNA virus of the Orthomyxovirus class. The virus contains a single stranded, negative sense, segmented (7-8 segments), RNA (ssNSRNA) genome.

While Influenza B has been overshadowed by Avian Influenza caused by the Influenza A H5N1 virus, the Influenza B virus remains a main cause of human illness and poses a risk to the young, elderly immuno-compromised individuals.

Current laboratory methods of detecting influenza B virus infections are based on conventional methods that involve antigen detection and isolation in cell culture. However, these techniques have their limitations. The cell culture techniques are too slow for it to be useful for diagnosis, while the antigen detection offers insufficient sensitivity and specificity. Thus, there are chances of having false positive results for influenza B that could be due to other subtypes. Moreover, some samples remain negative in spite of the clinical and epidemiological evidence of infection.

Molecular biology techniques like PCR and RT-PCR may be used for the detection of the virus. These techniques provide a method of rapid viral detection as well as subtype identification with defined primers. However, influenza viruses are prone to mutations. This suggests that there is a chance that any detection sequence chosen from the present sequence may not work. This may decrease the detection sensitivity and specificity of any of the detection methods currently available.

Accordingly, there is a need for a specific and/or sensitive detection method for influenza B virus infection.

### SUMMARY OF THE INVENTION

The present invention relates to oligonucleotide(s), method(s) and kit(s) for detecting the presence of an influenza virus in a biological sample or in a biological material amplified, isolated and/or purified from a biological sample.

Accordingly, the present invention provides an isolated oligonucleotide comprising a nucleotide sequence selected from the group consisting of: SEQ ID NO:1, SEQ ID NO:2, SEQ ID NO:3, and SEQ ID NO:4, fragment(s) thereof, derivative(s) thereof, mutation(s) thereof, and complementary sequence(s) thereof. In particular, there is provided an isolated oligonucleotide comprising essentially of an nucleotide sequence selected from the group consisting of: SEQ ID NO:1, SEQ ID NO:2, SEQ ID NO:3, and SEQ ID NO:4, fragment(s) thereof, derivative(s) thereof, mutation(s) thereof, and complementary sequence(s) thereof. More in particular, there is provided an isolated oligonucleotide consisting of a nucleotide sequence selected from the group consisting of: SEQ ID NO:1, SEQ ID NO:2, SEQ ID NO:3, and SEQ ID NO:4, fragment(s) thereof, derivative(s) thereof, mutation(s) thereof, and complementary sequence(s) thereof. In particular the SEQ ID NO:1, according to the invention may comprise, comprise essentially of, or consist of at least one nucleotide sequence selected from the group consisting of: SEQ ID NO:5, SEQ ID NO:6, SEQ ID NO:7 and SEQ ID NO:8. More in particular, the isolated oligonucleotide may be capable of binding to and/or being amplified from Influenza B virus.

The present invention also provides an amplicon amplified from an Influenza B virus using SEQ ID NO:1 as forward primer and SEQ ID NO:2 as reverse primer. The amplicon may comprise the sequence of SEQ ID NO:3. A probe comprising the sequence of SEQ ID NO:4 is capable of binding to the amplicon. The probe comprising the nucleotide sequence SEQ ID NO:4 may be used to further confirm the presence of the amplicon comprising at least the nucleotide sequence SEQ ID NO:3. In particular, the forward primer may comprise at least one nucleotide sequence selected from SEQ ID NO:5, SEQ ID NO:6, SEQ ID NO:7 and SEQ ID NO:8.

The present invention also provides a method of detecting the presence of an Influenza B virus in a biological sample, the method comprising the steps of:

(a) providing at least one biological sample;

(b) contacting at least one oligonucleotide with at least one nucleic acid in the biological sample, and/or contacting the oligonucleotide with at least one nucleic acid extracted, purified and/or amplified from the biological sample, wherein the oligonucleotide comprises at least one nucleotide sequence selected from the group consisting of:

SEQ ID NO:1, SEQ ID NO:2, SEQ ID NO:3, and SEQ ID NO:4, fragment(s) thereof, derivative(s) thereof, mutation(s) thereof, and complementary sequence(s) thereof;

and

(c) detecting any binding resulting from the contacting in step (b) whereby the virus is present when binding is detected.

In particular, the oligonucleotide used in the method of the invention may comprise at least a nucleotide sequence selected from the group consisting of: SEQ ID NOS:5 to 8.

The detecting in step (c) may comprise distinguishing between unbound oligonucleotide(s) and oligonucleotide(s) bound to the nucleic acid(s). The oligonucleotide(s) may be immobilized on a solid support. The solid support may comprise particles; the particles may comprise microbeads.

In particular, the oligonucleotide(s) according to the invention may be probe(s) and the method may comprise:

(i) providing at least one biological sample;

(ii) labeling at least one nucleic acid in the biological sample and/or labeling at least one nucleic acid extracted, purified and/or amplified from the biological sample with at least one reporter label;

(iii) immobilizing at least one probe to at least one particle (for example, at least one microbead) comprising at least one fluorescent dye;

(iv) contacting the probe with the nucleic acid to allow binding of the probe(s) and nucleic acid(s);

(v) identifying particles (microbeads) based on the fluorescent intensity of the fluorescent dye with a first laser light and detecting binding of nucleic acid(s) to probe(s) immobilized on (identified) microbead(s) with a second laser light based on the reporter label(s);

whereby the detection of binding of the nucleic acid(s) to probe(s) indicates the presence of Influenza B virus.

The microbead may comprise at least two fluorescent dyes. The two fluorescent dyes may be present in at least two different concentrations. The two different concentrations may impart a unique fluorescence intensity to the microbeads. The intensity of the two fluorescent dyes are capable of allowing one microbead to be distinguished from another microbead based on the fluorescent intensities of the two fluorescent dyes.

As an alternative, the labeling of the nucleic acid in step (ii) may be done after the contacting in step (iv) instead of before the contacting step.

According to a first aspect, the identification of particles (microbeads) and detection of nucleic acid(s) bound to the probe(s) may be carried out by Suspension Array Technology.

According to another aspect, the contacting in step (b) of the method of detecting may comprise contacting at least two oligonucleotides forming a primer pair to the nucleic acid and the step (c) of the method of detecting is by a polymerase chain reaction. The detecting then may be by determination of the molecular weight of at least one amplicon obtained from the polymerase chain reaction and/or by detection of binding of at least one probe to at least one amplicon.

For detecting by polymerase chain reaction, the primer pair may consist of a forward primer and a reverse primer which bind to the nucleic acid(s) and amplify at least one amplicon comprising the nucleotide sequence of SEQ ID NO:3. A probe comprising the nucleotide sequence of SEQ ID NO:4 is capable of binding to the amplicon. The polymerase chain reaction may be followed by electrophoresis for detection and/or purification of the amplicon. The reverse primer may be a primer comprising or consisting of the nucleotide sequence SEQ ID NO:2. The forward primer may be a primer comprising, comprising essentially, or consisting of the nucleotide sequence of SEQ ID NO:1. In particular, the forward primer comprises, comprises essentially, or consists of the nucleotide sequence selected from the group consisting of: SEQ ID NO:5, SEQ ID NO:6, SEQ ID NO:7 and SEQ ID NO:8.

According to the present invention, the contacting in step (b) and/or the binding in step (c) of the method of detecting may be for a time and under conditions sufficient for specific contacting and/or binding to occur between the oligonucleotide(s) or probe(s) and nucleic acid(s).

The biological sample may be from a human or non-human animal suspected to be infected with the Influenza B virus. The oligonucleotide(s) and/or the nucleic acid(s) may be labeled.

The present invention also provides a kit for the detection of Influenza B virus, the kit comprising at least one oligonucleotide comprising a nucleotide sequence selected from the group consisting of: SEQ ID NO:1, SEQ ID NO:2, SEQ ID NO:3, and SEQ ID NO:4, fragment(s) thereof, derivative(s) thereof, mutation(s) thereof, and complementary sequence(s) thereof. In particular, the oligonucleotide may comprise the nucleotide sequence of at least one nucleotide sequence selected from the group consisting of: SEQ ID NO:5, SEQ ID NO:6. SEQ ID NO:7 and SEQ ID NO:8, fragments thereof, complementary sequences, derivatives and mutations thereof. At least the one oligonucleotide may be labeled.

The kit may be for suspension array technology and may comprise at least one microbead, at least one fluorescent dye and/or at least one reporter label.

### BRIEF DESCRIPTION OF THE DRAWINGS

FIG. 1 shows a schematic diagram of the method of detecting the presence of a specific nucleic acid sequence using a specific probe and a Luminex® Suspension Array Technology. There are 8 essential steps shown in the diagram. Step 1 shows a probe with at least two fluorescent dyes. Half of the probe is striped designating the presence of one dye and the other half of the probe is a solid white designating the presence of the second dye. In step 2, a probe, shown as a thick zigzag line, is used to label each unique microbead. In step 3, the probe is contacted with at least one nucleic acid extracted, purified and amplified from a biological sample. The nucleic acid may be labeled with at least one reporter molecule. The labeled nucleic acid is shown as a thin zigzag line with the first label/reporter molecule represented as “V” attached to the thin zigzag line. In step 4, the microbeads with bound and/or hybridized nucleic acids are distinguished and/or separated from microbeads without bound and/or hybridized nucleic acids. A second label/reporter molecule which is fluorescent is then added as shown in step 5. The second fluorescent reporter molecule is represented as solid black arrows. In step 6, all the unbound fluorescent reporter molecules are removed and only the microbeads with bound and/or hybridized nucleic acids and bound fluorescent reporter molecules remain. As shown in step 7, the microbeads are individually identified by a first laser light “A” based on its unique identity of at least the two dyes that fluoresces at a specific wavelength. A second laser light “B” identifies and quantifies the amount of probe bound and/or hybridized to the labeled nucleic acids. which fluoresces at a different wavelength. In step 8, only probes bound and/or hybridized to the labeled nucleic acids which are excited by both laser lights are identified.

FIG. 2 shows the specificity and sensitivity of the oligonucleotides (probes) of the present invention. Bands in lanes 1-10 represent PCR products, amplified from nucleic acid sequences isolated and purified from biological samples, using the oligonucleotides of the present invention. The content of each biological sample is given in Table 1. The 271 bp product represents the amplified product of the Influenza Matrix 7 gene sequence. Lanes 11 to 20 show the specificity of the probes since there were no false positive results obtained despite the presence of several thousand copies of Influenza A (Flu A) and SARS viruses. The amplified sequences were also assayed by a suspension array method as shown in Table 1. The sensitivity of the oligonucleotides of the invention was demonstrated in lanes 9 and 10 where as few as two molecules or viral copies of Flu B isolated from the biological samples were detected in the PCR reaction.

### DETAILED DESCRIPTION OF THE INVENTION

Some terms used in the present description are defined hereunder. Well-known general molecular biology methods and techniques in the art not specifically described may be found in text books such as Sambrook et al. (ed.), Molecular Cloning: A Laboratory Manual, 3rd ed. , vol. 1-3, Cold Spring Harbor Laboratory Press, Cold Spring Harbor, N.Y., 2001. This book is also available as an online reference at http://www.molecularcloning.com/.

Definitions

Nucleotide: Includes, but is not limited to, a monomer that includes a base linked to a sugar, such as a pyrimidine, purine and/or synthetic analogs thereof, and/or a base linked to an amino acid, as in a peptide nucleic acid (PNA). A nucleotide is one monomer in a polynucleotide. A nucleotide sequence refers to the sequence of bases in a polynucleotide.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is preferably a polynucleotide of between 2 and 300 nucleotides in length. However, the length of an oligonucleotide is not limited to this size and the length may be less than 2 and more than 300 nucleotides. An oligonucleotide analog refers to moieties that function similarly to oligonucleotides but have non-naturally occurring portions. For example, oligonucleotide analogs may contain non-naturally occurring portions, such as altered sugar moieties and/or inter-sugar linkages, such as a phosphorothioate oligodeoxynucleotide. Functional analogs of naturally occurring polynucleotides may bind to RNA and/or DNA, and include peptide nucleic acid (PNA) molecules. Polynucleotide (also known as polynucleic acid) refers to RNA and/or DNA, as well as mRNA and/or cDNA corresponding to, or complementary to the RNA and/or DNA. According to the present invention, the term ‘polynucleotide’ also encompasses peptide nucleic acids (PNA). The term ‘gene’ comprises both sense and antisense strands of a polynucleotide which encodes a peptide, prepeptide, protein or marker, or refers to a vector or plasmid comprising such a polynucleotide, although usually, only the sequence of the sense strand is given. A fragment of a polynucleotide is a shortened length of the polynucleotide.

Nucleotide sequence ambiguity: In nucleotide sequences, a few nucleotides may change and/or mutate over time. In such changes and/or mutations, the original nucleotide is replaced and/or substituted by another, particularly one purine for another purine, and/or one pyrimidine for another pyrimidine. However, one purine may also substitute for a pyrimidine and vice versa. Where a nucleotide position is ambiguous and may be represented by one or more nucleotides standardized symbols or letters, well known to a person skilled in the art, are used, as given in the sequence listing of this application. Such symbols or letters (in either upper or lower case), proposed by the International Union of Pure and Applied Chemistry (IUPAC; Cornish-Bowden, 1985, Nucl. Acids Res. 13: 3021-3030) also corresponds to WIPO Standard ST.25 Appendix 2 Table 1, are as follows:

IUPAC nucleotide ambiguity codes

| Symbol | Meaning | Nucleic Acid |
|---|---|---|
| A | A | Adenine |
| C | C | Cytosine |
| G | G | Guanine |
| T | T | Thymine |
| U | U | Uracil |
| M | A or C | |
| R | A or G | |
| W | A or T | |
| S | C or G | |
| Y | C or T | |
| K | G or T | |
| V | A or C or G | |
| H | A or C or T | |
| D | A or G or T | |
| B | C or G or T | |
| X | G or A or T or C | |
| N | G or A or T or C | |

Mutation: A mutation is a change in the nucleic acid sequence of a length of nucleotides. A person skilled in the art will appreciate that small mutations, particularly point mutations of substitution, deletion and/or insertion has little impact on the stretch of nucleotides, particularly when the nucleic acids are used as probes. Accordingly, the oligonucleotide(s) according to the present invention encompasses mutation(s) of substitution(s), deletion(s) and/or insertion(s) of at least one nucleotide. Further, the oligonucleotide(s) and derivative(s) thereof according to the present invention, may also function as probe(s) and hence, any oligonucleotide(s) referred to herein also encompasses their mutations and derivatives.

The probes and/or primers of the invention may have any length of nucleotides. For example, the probes and/or primers may be from 9 to 50 nucleotides long. However, probes and/or primers having 2 to 8 nucleotides or more than 50 nucleotides may also be used. In particular, they may be from 12 to 30 nucleotides long. More in particularly, they may be from 15 to 25 nucleotides long. The sequences of the probes and/or primers used according to the present invention are given below.

Polynucleotides, probes and/or primers possess a certain sequence. Sequences of interest are listed according to the present invention. Two lengths of polynucleotides, probes and/or primers are said to possess the same sequence when they have the same sequence. However, according to the present invention, two sequences are also said to be the same if at least one specific probe and/or primer may bind to both sequences. According to the present invention, sequences are not thought to be the same if a probe and/or primer is unable to bind to both sequences under the same experimental and/or hybridization conditions.

When referring to a probe and/or primer, the term specific for (a target sequence) indicates that the probe and/or primer hybridizes under stringent conditions substantially only to the target sequence in a given sample comprising the target sequence.

Hybridization: The process wherein oligonucleotides and their analogs bind by hydrogen bonding, which includes Watson-Crick, Hoogsteen or reversed Hoogsteen hydrogen bonding, between complementary bases. Generally, nucleic acids consist of nitrogenous bases that are either pyrimidines (Cytosine (C), uracil (U), and thymine (T) or purines (adenine (A) and guanine (G)). These nitrogenous bases form hydrogen bonds consisting of a pyrimidine bound to a purine, and the binding of the pyrimidine to the purine is referred to as “base pairing.” More specifically, A will be bound to T or U, and G will be bound to C. “Complementary” refers to the base pairing that occurs between two distinct nucleic acid sequences or two distinct regions of the same nucleic acid sequence.

“Specifically hybridizable” and “specifically complementary” are terms which indicate a sufficient degree of complementarity such that stable and specific binding occurs between the oligonucleotide (or its analog) and the DNA and/or RNA target. The oligonucleotide and/or oligonucleotide analog need not be 100% complementary to its target sequence to be specifically hybridizable. An oligonucleotide and/or analog thereof is specifically hybridizable when binding of the oligonucleotide and/or analog thereof to the target DNA and/or RNA molecule interferes with the normal function of the target DNA and/or RNA, and there is a sufficient degree of complementarity to avoid non-specific binding of the oligonucleotide and/or analog thereof to non-target sequences under conditions in which specific binding is desired. For example, under physiological conditions in the case of in vivo assays. Such binding is referred to as “specific hybridization.” Hybridization conditions resulting in a particular degree of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization.

A person skilled in the art will appreciate that depending on the context, the terms “binding”, “hybridizing” or “hybridization” may be used interchangeably without giving rise to ambiguity.

The essential function of a primer and/or probe according to the present invention is to specifically hybridize to an influenza B virus nucleic acid, either an RNA or DNA, and not to cross-hybridize to other influenza nucleic acids and/or to nucleic acids of other viruses. Thus, a primer and/or probe “consists essentially of a nucleotide sequence if it includes that sequence and additional nucleotides that do not impair the ability of the primer and/or probe to specifically hybridize to an Influenza B virus nucleic acid under the conditions selected for performing a diagnostic assay according to the invention.

In vitro amplification: Techniques that increase the number of copies of a nucleic acid molecule in a sample and/or specimen. An example of amplification is the polymerase chain reaction, in which a biological sample collected from a subject is contacted with a pair of oligonucleotide primers (primer pairs), under conditions that allow for the hybridization of the primers to nucleic acid template in the sample. The primer pair may be thought to comprise an “upper” or “forward” primer and a “lower” or “reverse” primer each hydridizing to a (sense or antisense) strand of the DNA template sequence to be amplified. The primers are extended under suitable conditions, dissociated from the template, and then re-annealed, extended, and dissociated to amplify the number of copies of the nucleic acid. The product of in vitro amplification may be characterized by electrophoresis, restriction endonuclease cleavage patterns, oligonucleotide hybridization and/or ligation, and/or nucleic acid sequencing, using standard techniques.

Amplicon: The product of an in vitro nucleic acid amplification process is called an amplicon. The length of the amplicon may be derived by the start positions of the upper and lower primers, relative to a fixed reference of nucleotide position, usually to that of the upper (sense or coding) strand of the sequence to be amplified. For example, relative to the sense or coding strand, a theoretical 20-bp forward primer begins at position 100 and ends at position 120 and a 20-bp reverse primer starts at position 1000 and ends at position 980 (relative to the sense or coding strand). This primer pair will amplify a 900-bp amplicon from position 100 to 1000. The amplicon includes the forward and reverse primers. The length of an amplicon may also provide confirmation of successful hybridization of the primer sequences of the present invention.

Restriction site: A restriction site is a specific nucleic acid sequence recognized and cleaved by a restriction enzyme. An internal restriction site is a restriction site located within a particular nucleic acid sequence of interest.

Label: A chemical, moiety or molecule that allows detection of the label together with any molecule, surface or material to which the label is applied, attached, coupled, hybridized or bound to. Examples of labels include dyes, radiolabels, fluorescent labels, magnetic labels and enzymatic labels.

Reporter label: Any labeled reporter molecule known in the art. For example, at least one fluorescently labeled reporter molecule, like a fluorescent dye.

Biological sample: A sample of any tissue and/or fluid from at least one animal and/or plant. In particular, a sample of any tissue and/or fluid from at least a human.

Description

The present invention provides oligonucleotide(s), method(s) and/or a kit(s) for determining the presence of an influenza type and/or subtype virus in a biological sample or from biological material isolated, extracted, amplified and/or purified from a biological sample. In particular, the influenza type and/or subtype virus is the Influenza B virus.

The method of the present invention provides the use of at least one nucleic acid (or oligonucleotide) that recognizes sequences of the Influenza B virus to provide sensitive and/or specific means of detection. The detecting may be done according to any technique known in the art. For example, by polymerase chain reaction (PCR) and/or suspension array technique(s).

Oligonucleotides

The present invention provides at least one oligonucleotide. The oligonucleotide may be for use as probe(s) and/or primer(s) for detecting an Influenza virus. In particular, for detecting the Influenza B virus. The probe(s) and/or primer(s) of the invention may be of any length of nucleotides. For example, the probes and/or primers of the invention may be from 9 to 50 nucleotides long. However, probes and/or primers having 2 to 8 nucleotides or more than 50 nucleotides may also be used. In particular, they may be from 12 to 30 nucleotides long. More in particularly, they may be from 15 to 25 nucleotides long. The sequences of the probes and primers used under the present invention are given below.

The probes and/or primers of the present invention are designed to provide recognition of sequences in the Influenza B virus while allowing for single base mutations at specific locations.

In the present invention, SEQ ID NO:1, SEQ ID NO:2, SEQ ID NO:3, SEQ ID NO:4, SEQ ID NO:5, SEQ ID NO:6, SEQ ID NO:7 and/or SEQ ID NO:8, fragment(s) thereof, derivative(s) thereof, mutation(s) thereof, and complementary sequence(s) thereof are directed to the Matrix (M) or Segment 7 region of the Influenza B virus. When contacted and/or mixed with nucleic acids in a sample, the sequences of the present invention recognize, bind and/or hybridize to Influenza B sequences.

As an illustration, the present invention provides an amplicon amplified from an Influenza B virus using SEQ ID NO:1 as forward primer and SEQ ID NO:2 as reverse primer. The amplicon may comprise the nucleotide sequence of SEQ ID NO:3. A probe comprising the nucleotide sequence of SEQ ID NO:4 is capable of binding to the amplicon. The binding of probe comprising the nucleotide sequence of SEQ ID NO:4 to the amplicon may be considered a confirmation of the presence of Influenza B viral nucleic acids comprising the nucleotide sequence SEQ ID NO:3. In particular, the forward primer may comprise at least one nucleotide sequence selected from the group consisting of: SEQ ID NO:5, SEQ ID NO:6, SEQ ID NO:7 and SEQ ID NO:8.

A person skilled in the art will appreciate that the probe(s) and/or primer(s) may further comprise at least one label. A label is a chemical, moiety or molecule that allows detection of the label together with any molecule, surface or material to which the label is applied, attached, coupled, hybridized and/or bound to. Examples of labels include dyes, radiolabels, fluorescent labels, magnetic labels and enzymatic labels.

As such, the probes may also comprise other molecules to detect hybridized probes and target sequences. Examples of other molecules are biotin and avidin.

Such molecules allow recognition and/or binding of a label and/or reporter molecule to a nucleic acid sequence of interest.

Accordingly, the present invention provides primers, probes, oligonucleotides and nucleic acids that are labeled with suitable labels and/or reporter molecules.

In general, the method of the present invention comprises contacting the oligonucleotides of the invention as probes, primers and/or primer pairs with the sample and/or with nucleic acids obtained from the sample and then detecting any binding of the oligonucleotide(s) with the sample and/or nucleic acid(s) obtained therefrom.

Accordingly, the present invention provides a method of detecting the presence of an Influenza B virus in a biological sample, the method comprising the steps of:

(a) providing a biological sample;

(b) contacting at least one oligonucleotide with at least one nucleic acid in the biological sample, and/or contacting the oligonucleotide with at least one nucleic acid extracted, purified and/or amplified from the biological sample, wherein the oligonucleotide comprises at least one nucleotide sequence selected from the group consisting of:

SEQ ID NO:1, SEQ ID NO:2, SEQ ID NO:3, and SEQ ID NO:4, fragment(s) thereof, derivative(s) thereof, mutation(s) thereof, and complementary sequence(s) thereof;

and

(c) detecting any binding resulting from the contacting in step (b) whereby the virus is present when binding is detected.

In particular, the oligonucleotide may comprise at least one nucleotide sequence selected from the group consisting of: SEQ ID NOS:5 to 8.

The detecting in step (c) may comprise distinguishing between unbound oligonucleotide(s) and oligonucleotide(s) bound to the nucleic acid(s). The oligonucleotide(s) may be immobilized on a solid support. The solid support comprises at least one particle; the one particle may comprise at least one microbead.

Detection by Bead or Suspension Array Technology

The detecting may be done according to any technique known in the art. For example, the detecting may be done by Suspension Array Technology. For example, the Luminex® Suspension Array Technology (see web page www.upstate.com or U.S. Pat. No. 6,916,661, U.S. Pat. No. 6,939,720 or U.S. Pat. No. 6,514,295, the content of which is herein incorporated by reference). The Luminex® Suspension Array technology will be indicated as “luminex” for simplicity.

According to the present invention, the sample nucleic acid may optionally be amplified before being detected by the suspension array method. When used in suspension array techniques, one or more of the sequences of the present invention are bound to a solid support such as particles or microbeads.

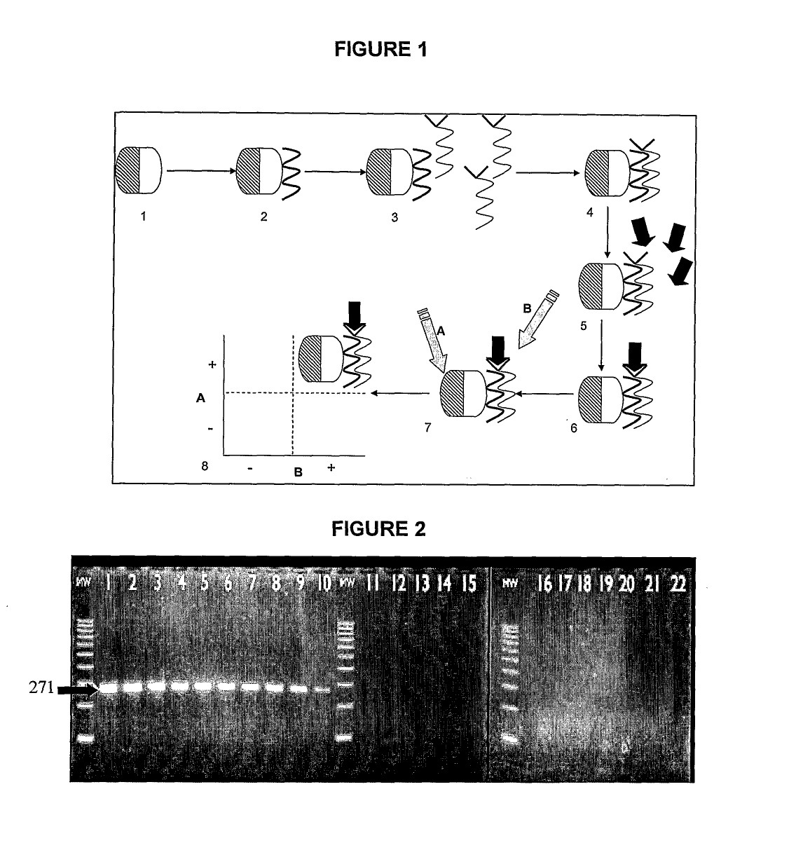

FIG. 1 shows a schematic diagram of the method of detecting the presence of a specific nucleic acid sequence using a specific probe and a Luminex® Suspension Array Technology. Each microbead is labeled with at least one fluorescent dye; in particular, with at least two dyes. Step 1 shows a probe with at least two fluorescent dyes. Half of the probe is striped designating the presence of one dye and the other half of the probe is solid white designating the presence of the second dye. The ratio of the striped region to the white region is not representative of the actual concentration ratio of the two dyes. The different concentrations of the two dyes give each microbead a unique fluorescent intensity when excited by a light of a specific wavelength. In step 2, a probe, shown as a thick zigzag line, is used to label each unique microbead. In step 3, the probe is contacted with at least one nucleic acid extracted, purified and amplified from a biological sample. The nucleic acid extracted, purified and amplified from a biological sample may be labeled with at least one reporter molecule. The labeled nucleic acid is shown as a thin zigzag line with the first label/reporter molecule represented as “V” attached to the thin zigzag line. In step 4, only the microbeads with bound and/or hybridized nucleic acids are distinguished and/or separated from microbeads without bound and/or hybridized nucleic acids for example, by centrifugation. A second label/reporter molecule which is fluorescent may then be added as given in step 5. The second fluorescent reporter molecule is represented as solid black arrows. In step 6, all the unbound fluorescent reporter molecules are removed and only the microbeads with bound and/or hybridized nucleic acids and bound fluorescent reporter molecules remain. As shown in step 7, the microbeads are individually identified by a first laser light “A” based on its unique identity of at least the two dyes that fluoresces at a specific wavelength. A second laser light “B” identifies and quantifies the amount of probe bound and/or hybridized to nucleic acids extracted, isolated and/or purified from the sample when at least one label and/or reporter molecule bound to either the probe or nucleic acid fluoresces at a different wavelength. In step 8, only probes bound and/or hybridized to nucleic acids extracted, isolated and/or purified from the sample which are excited by both laser lights are identified.

According to this technology, a liquid suspension array of up to 100 sets of 5.6 micron microbeads, each labelled with different ratios of concentrations of two spectrally distinct fluorophores, permitting each of the 100 sets of microbeads to be distinguished. Each microbead may be conjugated with a different capture molecule such as the probes of the present invention. The conjugated microbeads may then be mixed and incubated with samples in a micro titer plate to allow hybridizing of nucleic acids (for example, RNA purified from the sample, cDNA converted from the RNA from the sample or DNA amplicons amplified by PCR) extracted, isolated and purified from the sample. The nucleic acids in the sample may also be pre labeled with a first reporter molecule like biotin, for example. The biotin label allows for binding to another reporter molecule. The another reporter molecule may be fluorescent. The fluorescent molecule may be tagged to streptavidin which can bind to biotin., for example, a fluorescent streptavidin-R-phycoerythrin molecule.

Following incubation with fluorescently labeled reporter molecules, the contents of each well of the micro titer plate are analysed in a luminex where the microbeads are aligned in single file through a flow cell. Two lasers excite the microbeads individually. A first (red) classification laser excites the dyes in each microbead to give off fluorescent signals, the intensities of the fluorescent signals identify each microbead's spectral address or identity. A second (green) reporter laser excites the reporter molecule associated with the microbead or sample, which allows quantitation of the captured sample nucleic acids. The fluorescent signals are then simultaneously recorded for each microbead, translating the signals into data for each bead-based assay. This analysis step may also distinguish microbeads with captured sample nucleic acids from the beads without captured sample nucleic acids.

Accordingly, the present invention provides detecting of binding of the oligonucleotide(s) and the sample and/or nucleic acid(s) by suspension array technology. The suspension array technology may comprise:

(i) providing at least one biological sample;

(ii) labeling at least one nucleic acid in the biological sample and/or extracted, purified and/or amplified from the biological sample with at least one reporter label;

(iii) immobilizing at least one probe to at least one microbead comprising at least one fluorescent dye;

(iv) contacting the probe with the labeled nucleic acid to allow binding of the probe(s) and nucleic acid(s);

(v) identifying microbead(s) based on the fluorescent intensity of the fluorescent dye with a first laser light and detecting binding of nucleic acid(s) to probe(s) immobilized on (identified) microbead(s) with a second laser light based on the reporter label(s);

whereby the detection of binding of the nucleic acid(s) to probe(s) indicates the presence of the Influenza B virus.

The reporter label bound to the nucleic acid may also be a fluorescent dye or a label capable of binding a fluorescent molecule.

The microbead may comprise at least one fluorescent dye, in particular at least two fluorescent dyes. The two fluorescent dyes are capable of allowing one microbead to be distinguished from another microbead based on the fluorescent intensities of the two fluorescent dyes.

As an alternative, the labeling of the nucleic acid in step (ii) may be done after the contacting in step (iv) instead of before the contacting step.

Detection by Polymerase Chain Reaction

According to another aspect, the method of the invention may comprise performing an amplification step. For example, the method may comprise detection means comprising a polymerase chain reaction (PCR) format using one or more probe/primer or primer pairs.

According to the present invention, the oligonucleotide may be one of the two oligonucleotides forming a primer pair and the step (c) of detecting may be by a polymerase chain reaction.

The detecting may be by detection of at least one label released by the polymerase chain reaction such as that released by the hybridization of at least one probe and/or primer during real time polymerase chain reaction, thus detecting the number of copies of nucleic acids present in the sample. The detecting may be by determination of the molecular weight of at least one amplicon obtained from the polymerase chain reaction and/or the detecting may be by detection of binding of at least one probe to the least one amplicon. The method may be performed, for example, with PCR by amplifying nucleic acids present in the sample using at least one forward primer and at least one reverse primer (a primer pair) selective for the region of the Influenza B genome such as the Matrix gene to obtain an amplification product or amplicon. Any binding, hybridization and/or amplification product may then be detected, for example, by determining the length and/or molecular weight of the amplification product in nucleotides, either by a chromatographic method and/or by a gel electrophoretic method. The presence of an amplification product having a length in nucleotides that is the sum of the forward primer length, the reverse primer length and the separation length between the primers indicates the presence of the Matrix nucleic acid of the Influenza B virus in the sample.

A person skilled in the art will appreciate that the probes of the present invention may also be used as a primers for PCR detection as they demarcate a stretch of nucleic acids that may be amplified. Accordingly, the terms primers and/or probes may be referred to interchangeably depending on the context and/or detection method wherein they are used. Real-time PCR detection according to the present invention may be performed using PCR platforms such as the Roche LightCycler™, the Stratagene Real-time PCR system, the Applied Biosystems ABI 7000 real time PCR analyzer or any other suitable detection platform.

Alternatively, the product may be detected with PCR using a hybridization probe, for example using real-time fluorescent detection such as the Taqman™ system (Applied Biosystems, Foster City, Calif.) wherein a fluorescent label attached to the probe is released by the polymerase when the probe is bound and/or hybridized to the target sequence and extended by the polymerase. The amount of label released thus gives an indication of the quantity of target sequences present in the sample.

When PCR methods are used to detect hybridization, the hybridization probe may comprise a nucleotide sequence that is the same as that of a portion of the amplification product that would be obtained using the amplification primers selected and an Influenza B virus genomic nucleic acid as a template. The hybridization probe may be of any suitable length.

When used in PCR, a pair of sequences of the present invention may function as the forward (or upper) primer and lower (or reverse) primers for a stretch of Influenza B sequence. These primers then allow the stretch of Influenza B sequence to be amplified by a PCR method. The amplicon thus obtained may be identified by its size (length or molecular weight) from gel electrophoresis and/or from a probe binding to the amplicon.

As an illustration, the primer pair may consist of a forward primer comprising the nucleotide sequence of SEQ ID NO:1 and a reverse primer comprising the nucleotide sequence of SEQ ID NO:2 which bind to the nucleic acid(s) and amplify at least one amplicon comprising the sequence of SEQ ID NO:3. A probe comprising the sequence of SEQ ID NO:4 is capable of binding to the amplicon. The polymerase chain reaction may be followed by electrophoresis for detection and/or purification of the amplicon. In particular, the forward primer may comprise at least one nucleotide sequence selected from the group consisting of: SEQ ID NO:5, SEQ ID NO:6, SEQ ID NO:7 and SEQ ID NO:8.

According to the method of the present invention, the contacting and/or the binding may be for a time and under conditions sufficient for specific contacting and/or binding to occur between the oligonucleotide(s) or probe(s) and nucleic acid(s).

The biological sample may be from a human or non-human animal suspected to be infected with the Influenza B virus. The oligonucleotide and/or the nucleic acid may be labeled.

The present invention also provides a kit for the detection of Influenza B virus, the kit comprising at least one oligonucleotide selected from the group consisting of: SEQ ID NO:1, SEQ ID NO:2, SEQ ID NO:3, and SEQ ID NO:4, fragment(s) thereof, derivative(s) thereof, mutation(s) thereof, and complementary sequence(s) thereof. More in particular, the oligonucleotide comprise at least one nucleotide sequence selected from the group consisting of: SEQ ID NOS:5 to 8.

The oligonucleotide in the kit may be labeled. The kit may be for detecting the presence of an Influenza B virus. The kit may be for use with a biological sample, where the biological sample may be from a human or non-human animal suspected to be infected with an Influenza B virus. A kit according to the invention may optionally include a positive control nucleic acid, for example a nucleic acid, or at least a portion thereof, comprising the M region of the influenza B virus, as either RNA or DNA. The kit may further comprise information or instructions pertaining to its use.

The kit may be for suspension array technology and may comprise at least one microbead, at least one fluorescent dye and/or at least one reporter label.

The examples provide a simple, sensitive and/or specific diagnostic test. By use of the probes and/or primers described herein, the method and/or kits are made more sensitive and/or specific than the detection methods of the prior art. A person skilled in the art will appreciate that such tests may be by bead-suspension array technology. Alternatively, such tests may be by a one step PCR method, a two step PCR or real time PCR.

Having now generally described the invention, the same will be more readily understood through reference to the following examples which are provided by way of illustration, and are not intended to be limiting of the present invention.

### Examples

### Example 1

### Materials and Methods used in the Examples

Standard molecular biology techniques known in the art and not specifically described were generally followed as described in Sambrook and Russel, Molecular Cloning: A Laboratory Manual, Cold Springs Harbor Laboratory, N.Y. (2001).

Standards

A 10-fold dilution of each stock virus was prepared in serum obtained from a healthy volunteer. RNA was extracted using the QIAGEN Viral RNA Kit (QIAGEN GMbH, Germany).

Human patient specimens were obtained from patients diagnosed with, or suspected to be infected with influenza B at Tan Tock Seng Hospital in Singapore.

Virus isolation was performed on a serum specimen of Influenza B cases. RNA was directly extracted from the specimen using a Qiagen QIAamp viral RNA extraction kit (catalog no. 52906) according to the manufacturer's instructions.

RNA was extracted from infected patients in a manner understood by those in the art and treated with Qiagen RLT buffer, a proprietary product that contains guanidine and β-mercaptoethanol.

Other Viruses

RNA was directly extracted from virus sample stock vials obtained from the American Type Culture Collection (ATCC; VA, USA) using the QIAGEN viral RNA Mini Kit (QIAGEN GMbH, Germany) according to the manufacturer's instructions. It would be understood by those in the art that RNA isolated from any non-influenza virus would be sufficient to provide a suitable negative control.

MRC-5 Cell Line

Total RNA was extracted directly from the normal diploid human fibroblast cell line MRC-5 (ATCC CCL171) using a Qiagen RNA extraction kit (catalog no. 74104) and RNA was quantified using a spectrophotometer.

Detection

RNA was extracted from samples suspected to contain Influenza B RNA as assessed by known methods. The RNA was then converted to cDNA using a reverse transcriptase or any other method known in the art. A sample mixture was converted into cDNA in a typical manner using a 1st Strand cDNA Synthesis Kit for RT-PCR (Roche, Basel, Switzerland, catalog no. 1 483 188).

Materials for Suspension Array

Suspension array apparatus and microbeads were purchased from Luminex Corporation. Streptavidin-R-phycoerythrin conjugate was purchased from Invitrogen/Molecular Probes (Eugene, Oreg.). A Luminex 100 system, from Luminex Corporation (Austin, Tex.) was used for the detection of beads by suspension array technology.

### Example 2

### Probes and Primers

While the probes/primers of the present invention are designed to detect the Influenza B virus by suspension array technology, a person skilled in the art will appreciate that the following sequences may also be used in other suitable detection methods for Influenza B as well, such as in situ hybridization, nuclease protection assay, etc. All primers described here are designed based on the sequences provided by NCBI Influenza Virus Sequence Database. (http://www.ncbi.nlm.nih.gov/genomes/influenza/list.cgi)

Probes for Matrix Gene

The following probes (SEQ ID NOS:1 to 8) were designed as generic probes to detect the Influenza B virus by recognizing and/or demarcating a 271 base pair (bp) portion of the M gene (Matrix gene or segment 7).

The 271 bp portion corresponds to nucleotides 233-503 of the BM2 gene as represented by NCBI Accession number AB120273 incorporated herein for reference (Matsuzaki et al, 2004).

M Upper/Forward Probe/Primer

| | |
|---|---|
| 5′- TCATCACAGARCCCYTATCAG -3′ | (SEQ ID NO: 1) |
| R = A/G | |
| Y = C/T | |

In particular, the sequence is

(SEQ ID NO: 5)
5′-TCATCACAGAACCCCTATCAG-3′
(SEQ ID NO: 6)
5′-TCATCACAGAACCCTTATCAG-3′
(SEQ ID NO: 7)
5′-TCATCACAGAGCCCCTATCAG-3′
(SEQ ID NO: 8)
5′-TCATCACAGAGCCCTTATCAG-3′
M Lower/Reverse Probe/Primer
(SEQ ID NO: 2)
5′-GATCTCGCTGCTCTGCTATGAG-3′
M Amplicon
(SEQ ID NO: 3)
5′-TCATCACAGAGCCCTTATCAGGAATGGGAACAACAGCAACAAAAA
AGAAAGGCCTGATTCTGGCTGAGAGAAAAATGAGAAGATGTGTGAGCT
TTCATGAAGCATTTGAAATAGCAGAAGGCCATGAAAGCTCAGCGCTAC
TATACTGTCTCATGGTCATGTACCTGAATCCTGGAAATTATTCAATGC
AAGTAAAACTAGGAACGCTCTGTGCTTTGTGCGAGAAACAAGCATCAC
ATTCACACAGGGCTCATAGCAGAGCAGCGAGATC-3′
M Probe
(SEQ ID NO: 4)
5′-TTGTTGCTGTTGTTCCCATTCCTG-3′

For SEQ ID NO: 1, nucleotide sequence ambiguity gives rise to a possible combination of at least 4 variations of the sequence under the present invention.

A person skilled in the art will appreciate that the probes and/or primers above may be labeled so as to allow detection of the probe and/or primer by any suitable method. The label may be any chemical, moiety or molecule that allows detection of the label together with any molecule, surface or material to which the label is applied, attached, coupled, hybridized or bound to. Examples of labels include dyes, radiolabels, fluorescent labels, magnetic labels and enzymatic labels.

### Example 3

### Influenza B Virus Detection by Suspension Array Technology

Labeling of Microbeads

The above probes were synthesized by any commercially-available oligonucleotide synthesizer. These probes were then coupled to polystyrene-methacrylate Luminex microbeads (Luminex Corporation, Tex., USA) approximately 5.6 microns in diameter pre-stained with red and orange fluorophores. The probes were coupled and/or immobilized to the microbeads via the microbeads' surface carboxyl groups. After coupling, the microbeads were mixed to form a multiplexed set. For comprehensive screening of samples, all combinations of the probe sequences of the present invention were included in the multiplexed set. Microbeads with suitable control sequences immobilized to them may also be included in the multiplexed set.

Hybridization

Nucleic acid extracted, purified and isolated from the biological samples were amplified by PCR before being contacted with the multiplexed set of microbeads immobilized with the probes. For control, Influenza A subtype viral nucleic acids (obtained from Australian Animal Health Laboratory (AAHL)) as well as non-template control transfer RNA (NTC tRNA), estimated by dilution of stock supplies, were used. Hybridization was allowed to occur in a 1× TMAC Hybridisation Solution (3M TMAC, 0.1% Sarkosyl, 50 mM Tris HCl (pH 8.0), 4 mM EDTA (pH 8.0) for 40 minutes at 60° C. after the initial denaturation at 95° C. for 10 minutes.

Following hybridization and washing to remove unbound nucleotides, 1/500 dilution of SA-PE (streptavidin-R-phycoerythrin conjugate (1 mg/ml), Invitrogen (Cat: S866)) was added and incubated at 52° C. for 5 min. The presence of target nucleic acid sequences in the sample was analyzed and detected in a Luminex 100 system.

Results

The probes of the present invention, in combination with a suspension array technology provide single-molecule sensitivity levels for detection of Influenza B (Flu B) in infected samples. As indicated in Table 1, the method of the present invention was able to detect as few as two molecules or viral copies (Locations B2 and C2, for example) of Flu B isolated from the biological samples. Despite the high content of non-Influenza B viruses (20,000 copies) present, the probes of the current invention did not show hybridization to the nucleic acid of these viruses. This specificity of the probes can be seen in the very low background counts obtained by the suspension array method (Table 1). The probes of the invention identified the presence of Flu B with very high sensitivity when used with this technology. While the Flu A, SARS viruses and the negative control NTC tRNA gave background counts (less than the threshold count of 150), Flu B nucleic acids isolated from the biological samples gave counts that were at least 10 fold more than these negative controls. Influenza A subtype viruses, for example, all registered a count less than the threshold count of 150 (column Flu B M Probe) compared to counts of over 1,000 for the Influenza B samples.

TABLE 1
Sensitivity and Specificity of Influenza B Detection Analysis by Suspension Array Technology

| | | | Probe specificity−> FluA (HN51) | | FluA all | FluA (HN51) | FluB | |
|---|---|---|---|---|---|---|---|---|
| | Gel lane | | Est. viral copies | Flu A-H | Flu A-M | Flu A-N | Flu B-M | Total |
| Location | no. | Sample | per rxn | Probe | Probe | Probe | Probe | events |
| A1 | 1 | Flu B (−5)1 | 200 | 84 | 89 | 91 | 3748 | 437 |
| B1 | 2 | Flu B (−5)2 | 200 | 47 | 72 | 71 | 3638 | 468 |
| C1 | 3 | Flu B (−5.5)1 | 70 | 81 | 67 | 100 | 3790 | 438 |
| D1 | 4 | Flu B (−5.5)2 | 70 | 70 | 75 | 80 | 3635 | 444 |
| E1 | 5 | Flu B (−6)1 | 20 | 72 | 70 | 78 | 3691 | 513 |
| F1 | 6 | Flu B (−6)2 | 20 | 81 | 48 | 88 | 3231 | 487 |
| G1 | 7 | Flu B (−6.5)1 | 7 | 81 | 65 | 73 | 3242 | 428 |
| H1 | 8 | Flu B (−6.5)2 | 7 | 63 | 73 | 83 | 2400 | 437 |
| A2 | 9 | Flu B (−7)1 | 2 | 73 | 64 | 81 | 1846 | 510 |
| B2 | 10 | Flu B (−7)2 | 2 | 47 | 104 | 92 | 1161 | 435 |
| C2 | 11 | Flu A H5N1(−3)1 | 20,000 | 75 | 70 | 68 | 65 | 415 |
| D2 | 12 | Flu A H5N1(−3)2 | 20,000 | 89 | 68 | 96 | 91 | 433 |
| E2 | 13 | Flu A H7N7(−3)1 | 20,000 | 68 | 77 | 71 | 59 | 430 |
| F2 | 14 | Flu A H7N7(−3)2 | 20,000 | 45 | 81 | 58 | 63 | 468 |
| G2 | 15 | Flu A H7N3(−3)1 | 20,000 | 84 | 70 | 68 | 61 | 459 |
| H2 | 16 | Flu A H7N3(−3)2 | 20,000 | 68 | 51 | 48 | 71 | 432 |
| A3 | 17 | Flu A H5N3(−3)1 | 20,000 | 60 | 56 | 103 | 73 | 423 |
| B3 | 18 | Flu A H5N3(−3)2 | 20,000 | 77 | 60 | 73 | 60 | 454 |
| C3 | 19 | SARS (−3) 1 | 20,000 | 69 | 52 | 77 | 102 | 455 |
| D3 | 20 | SARS (−3) 2 | 20,000 | 53 | 87 | 81 | 48 | 562 |
| E3 | 21 | NTC, tRNA 1 | 0 | 56 | 70 | 92 | 63 | 551 |
| F3 | 22 | NTC, tRNA 2 | 0 | 58 | 77 | 44 | 85 | 441 |

While more than one probe to each gene are provided, a person skilled in the art will appreciate that only one probe is needed for identification of the Influenza B virus. Use of more than one probe increases the confidence of identifying the virus.

### Example 4

### Influenza B Virus Detection by RT-PCR

The primers may also be used to detect Influenza B virus in gel-based RT-PCR. RNA from the samples may be converted to cDNA and amplified using the primers in a PCR reaction. The amplified products may then be detected by agarose gel electrophoresis or by detection of a probe (for example, SEQ ID NO:4 or a fragment thereof) hybridized to the amplicon.

Primer Pair for Matrix Gene for Influenza B

The sequences of SEQ ID NO:1, SEQ ID NO: 5, SEQ ID NO:6, SEQ ID NO:7, SEQ ID NO:8, were used as the forward primer and the SEQ ID NO:2 was used as the reverse primer to amplify amplicons or fragments possessing the sequence of SEQ ID NO:3 or fragments thereof.

Protocol

Protocol for One Step RT-PCR

(a) Sample Preparation

In a RNase-Free Eppendorf tube (0.5 ml or 0.2 ml size), add the following reagents per test/per reaction:

| Tube No. | Description | 50 μl/Rxn | 20 μl/Rxn |
|---|---|---|---|
| Tube 1 | Primer Mix (3 primer sets) | 2.0 μl | 0.8 μl |
| From | Qiagen 5x buffer | 10.0 μl | 4.0 μl |
| Qiagen | Qiagen dNTP Mix | 2.0 μl | 0.8 μl |
| Kit | Qiagen Enzyme Mix | 2.0 μl | 0.8 μl |
| | RNase-free Water | 29.0 μl | 11.6 μl |
| Tube 2 or | RNA Sample | 5.0 μl | 2.0 μl |
| test sample | | | |
| | Total Volume | 50.0 μl | 20.0 μl |

A positive and/or negative control may be included for validating the results. The positive control being in the form of a cDNA comprising SEQ ID NO:3 (which is the 271 bp portion corresponding to nucleotides 233-503 of the Influenza B M2 gene as represented by NCBI Accession number AB120273) or a fragment thereof and the negative control being any RNA isolated from a non-influenza type virus, or even from another Influenza virus such as Influenza A virus.

(b) Thermal Cycling Protocol—A

Thermal cycling conditions for three-blocks type PCR cycler such as RoboCycler® by Stratagene:

| | | | | |
|---|---|---|---|---|
| | Temp | | Num of | |
| Step | (° C.) | Duration | Cycle(s) | Step |

| | | | | | |
|---|---|---|---|---|---|
| 1 | 60 | 30 | mins | 1 | Reverse |
| | | | | | transcription |
| 2 | 95 | 15 | mins | 1 | Initial |
| | | | | | denaturation |
| 3 | 95 | 45 | secs | 42 | Denaturation |
| | 59 | 76 | secs | | Annealing |
| | 72 | 45 | secs | | Extension |
| 4 | 72 | 3 | mins | 1 | Final |
| | | | | | Extension |

(c) Thermal Cycling Protocol—B

Thermal cycling conditions for one-block type PCR cycler such as Px2 Thermal Cycler by Thermo Electron.

| | | | | |
|---|---|---|---|---|
| | Temp | | No. of | |
| Step | (° C.) | Duration | Cycle(s) | Step |

| | | | | | |
|---|---|---|---|---|---|
| 1 | 60 | 30 | mins | 1 | Reverse |
| | | | | | transcription |
| 2 | 95 | 15 | mins | 1 | Initial |
| | | | | | denaturation |
| 3 | 95 | 15 | secs | 42 | Denaturation |
| | 59 | 25 | secs | | Annealing |
| | 72 | 24 | secs | | Extention |
| 4 | 72 | 3 | mins | 1 | Final Extention |

(d) Termination of PCR Reaction

(This step is optional).

(1) Add 30 ul of Chloroform/Tube. Vortex mix for 5 secs

(2) Centrifuge for 2 mins. (Top=Aqueous phase, Bottom=Organic phase)

(e) Electrophoresis

Resolve the above product by DNA gel electrophoresis.

(1) DNA electrophoresis with 3% agarose gel

(2) Use voltage, for example, at 100V for 30 mins.

(3) Stain with ethidium bromide, destain and visualize under UV light.

(f) Validating Results

The expected amplicon product size is 271 bp. A labeled probe with SEQ ID NO:4 or fragment thereof will bind to the amplicon and be detected as confirmation. FIG. 2 shows the 271 bp amplification product of the above nucleic acids extracted from patient samples using the primers and probes of the present invention.

### Example 5

### Detection Kits

The detection method and/or kit of the present invention lies generally in use of a set of probes that are specific for segment 7 or Matrix gene of the Influenza B virus genome for detection of presence of the virus.

To carry out the present the invention, the following kits may be used.

Kit 1 for Use with PCR Technology

For general detecting and determining of the presence of Influenza B virus in a sample, a kit comprising at least one sequence according to SEQ ID NOS:1 to 8 for use as primers, probes and/or control sequences is provided.

The kit may comprise SEQ ID NO:1 and SEQ ID NO:2 for use as a primer pair. The kit may optionally comprise SEQ ID NO:3 as a positive control and/or SEQ ID NO:4 as a probe for the amplicon. This kit is optimized to detect 1 to 100 molecules of the viral RNA in 5 μl of test sample. Amplified products may be detected by agarose gel electrophoresis. The entire procedure is performed in one step according to the protocol of Example 3.

Kit 2 for Use in Suspension Array Technology

For general detecting and determining of the presence of Influenza B virus in a sample, a kit comprising microbeads with at least one nucleic acid sequence of SEQ ID NO:1 to 8 immobilized to the microbeads are provided.

The kit may optionally further comprise a suitable dye for quantitation by the Luminex system, information pertaining to use of the kit and positive and negative controls. The entire procedure is performed according to the protocol of Example 3. The kits of the invention may also further comprise information and/or instructions pertaining to their use.

A person skilled in the art will recognize that the above examples may be combined to practice the invention. For example, any nucleic acids bound to probes on microbeads may be amplified by PCR in situ while still bound to the beads before being subject to detection. Further, the PCR may be real time PCR to further quantify the amount of target nucleic acids originally in the sample. Labels to allow detection may be conjugated to either the target sequences or to the primers or probes before or after binding, hybridization or amplification.

As the invention relates generally to the detection and/or diagnosis of the presence of the Influenza B in a biological sample using the nucleic sequences of the present invention (SEQ ID NOS:1-8), a person skilled in the art will recognize that the sequences of the present invention may be manipulated in any number of ways and methods. These include replication, amplification and cloning into vectors and transformation into suitable cells or organisms, as long as the sequences, their complementary sequences, RNA or DNA copies thereof, and/or fragments thereof, can be used to obtain the primers and probes of the present invention for the detection and diagnosis of the Influenza B virus.

Although the present invention has been described in detail with reference to examples above, it is understood, that various modifications may be made without departing from the spirit of the invention. Accordingly, the invention is only limited by the following claims. Any and all cited patents, patent applications and publications referred to in this application is herein incorporated by reference in their entirety.

### REFERENCES

Matsuzaki et al. (2004). Genetic diversity of influenza B virus: the frequent reassortment and cocirculation of the genetically distinct reassortant viruses in a community. J. Med. Virol. 74(1), 132-140.

U.S. Pat. No. 6,916,661

U.S. Pat. No. 6,939,720

U.S. Pat. No. 6,514,295

1. An isolated oligonucleotide comprising at least one nucleotide sequence selected from the group consisting of: SEQ ID NO:1, SEQ ID NO:2, SEQ ID NO:3, and SEQ ID NO:4, derivative(s) thereof, mutation(s) thereof, and complementary sequence(s) thereof.

2. The isolated oligonucleotide according to claim 1, wherein the isolated oligonucleotide comprises essentially of at least one nucleotide sequence selected from the group consisting of: SEQ ID NO:1, SEQ ID NO:2, SEQ ID NO:3, and SEQ ID NO:4 derivative(s) thereof, mutation(s) thereof, and complementary sequence(s) thereof.

3-5. (canceled)

6. The isolated oligonucleotide according to claim 1, wherein the oligonucleotide comprises at least one nucleotide sequence selected from the group consisting of: SEQ ID NO:6, SEQ ID NO:7, and SEQ ID NO:8.

7. The isolated oligonucleotide according to claim 1, wherein the oligonucleotide comprises nucleotide sequence SEQ ID NO:5.

8. The isolated oligonucleotide according to claim 1, wherein the oligonucleotide is capable of binding to and/or being amplified from Influenza B virus.

9. An amplicon amplified from Influenza B virus using at least one forward primer comprising the nucleotide sequence of SEQ ID NO:1 and at least one reverse primer comprising the nucleotide sequence of SEQ ID NO:2.

10. The amplicon according to claim 9, wherein the forward primer comprises at least one nucleotide sequence selected from the group consisting of: SEQ ID NO:5. SEQ ID NO:6, SEQ ID NO:7, and SEQ ID NO:8

11. The amplicon according to claim 9, wherein the amplicon comprises the nucleotide sequence of SEQ ID NO:3.

12. The amplicon according to claim 9, wherein at least one probe comprising the nucleotide sequence of SEQ ID NO:4 is capable of binding to the amplicon.

13. A method of detecting the presence of an Influenza B virus in a biological sample, the method comprising the steps of:
(a) providing at least one biological sample;
(b) contacting at least one oligonucleotide with at least one nucleic acid in the biological sample, and/or contacting the oligonucleotide with at least one nucleic acid extracted, purified and/or amplified from the biological sample, wherein the oligonucleotide comprises at least one nucleotide sequence selected from the group consisting of:
SEQ ID NO:1, SEQ ID NO:2, SEQ ID NO:3, and SEQ ID NO:4, derivative(s) thereof, mutation(s) thereof, and complementary sequence(s) thereof;
and
(c) detecting any binding resulting from the contacting in step (b) whereby the virus is present when binding is detected.

14. The method according to claim 13, wherein the oligonucleotide comprises at least one nucleotide sequence selected from the group consisting of: SEQ ID NO:5. SEQ ID NO:6, SEQ ID NO:7, and SEQ ID NO:8.

15-16. (canceled)

17. The method according to claim 13, wherein the oligonucleotide(s) are probe(s) and the method comprises:
(i) providing at least one biological sample;
(ii) labeling at least one nucleic acid in the biological sample and/or at least one nucleic acid extracted, purified and/or amplified from the sample with at least one reporter label;
(iii) immobilizing at least one probe to at least one microbead comprising at least one fluorescent dye;
(iv) contacting the probe with the nucleic acid to allow binding of the probe(s) and nucleic acid(s);
(v) identifying microbeads based on the fluorescent intensity of the fluorescent dye with a first laser light and detecting binding of nucleic acid(s) to probe(s) immobilized on microbead(s) with a second laser light based on the reporter label(s);
whereby the detection of binding of the nucleic acid(s) to probe(s) indicates the presence of Influenza B virus.

18-19. (canceled)

20. The method according to claim 17, wherein the labeling of at least the one nucleic acid in step (ii) is done after the contacting in step (iv).

21. The method according to claim 13, wherein the step (c) of detecting is carried out by using Suspension Array Technology.

22. The method according to claim 13, wherein the contacting in step (b) comprises contacting at least two oligonucleotides forming a primer pair to the nucleic acid and the step (c) of detecting is by a polymerase chain reaction.

23-24. (canceled)

25. The method according to claim 22, wherein the primer pair binds to the nucleic acid(s) and amplifies at least one amplicon comprising the sequence of SEQ ID NO:3, the primer pair comprising at least one forward primer comprising the nucleotide sequence SEQ ID NO:1 and at least one reverse primer comprising the nucleotide sequence SEQ ID NO:2.

26. The method according to claim 22, wherein the forward primer comprises at least the nucleotide sequence selected from the group consisting of: SEQ ID NO:5. SEQ ID NO:6, SEQ ID NO:7, and SEQ ID NO:8.

27. The method according to claim 22, wherein a probe comprising the nucleotide sequence of SEQ ID NO:4 is capable of binding to the amplicon.

28-32. (canceled)

33. A kit for the detection of Influenza B virus, the kit comprising at least one oligonucleotide comprising a nucleotide sequence selected from the group consisting of:
SEQ ID NO:1, SEQ ID NO:2, SEQ ID NO:3, and SEQ ID NO:4 derivative(s) thereof, mutation(s) thereof, and complementary sequence(s) thereof.

34. The kit according to claim 33, wherein the oligonucleotide comprises at least one nucleotide sequence selected from the group of: SEQ ID NO:5. SEQ ID NO:6, SEQ ID NO:7, and SEQ ID NO:8.

35-36. (canceled)
